(19) United States
(12) Patent Application Publication
(10) Pub. No.: US 2022/0168456 A1
(43) Pub. Date: Jun. 2, 2022

(54) Sterilization Box Assembly

(71) Applicant: Leisa Jones, Jackson, MS (US)

(72) Inventor: Leisa Jones, Jackson, MS (US)

(21) Appl. No.: 17/108,313

(22) Filed: Dec. 1, 2020

(51) Int. Cl. A61L 2/26; A61L 2/10

(52) U.S. Cl. CPC A61L 2/26; A61L 2/10; A61L 2202/11; A61L 2202/121; A61L 2202/122

(57) ABSTRACT

A sterilization box assembly for sterilizing personal objects includes a box that can have a variety of objects positioned therein. A lid is hingedly coupled to the box for opening and closing the box. A plurality of light emitters is each coupled to the lid and each of the light emitters emits light into the box. Each of the light emitters has an operational frequency in the ultra-violet spectrum of light to kill bacteria and sterilize the objects in the box. A lever extends through the outer wall of the box wherein the lever is configured to be manipulated by a user. The lever is positionable between an on position and an off position for turning the light emitters on and off.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to sterilization devices and more particularly pertains to a new sterilization device for sterilizing personal objects. The sterilization device employed ultraviolet let and an audible alert that to alert a user that sufficient time has passed to sterilized the personal objects.

### (2) Description of Related Art Including Information Disclosed Under 37 CFR 1.97 and 1.98

The prior art relates to sterilization devices including a variety of boxes that have a source of ultraviolet radiation integrated therein to sterilize objects contained in the box. In no instance does the prior art disclose a box that includes a source of ultraviolet radiation and an audible alert to alert a user when a sterilization cycle has completed.

### BRIEF SUMMARY OF THE INVENTION

An embodiment of the disclosure meets the needs presented above by generally comprising a box that can have a variety of objects positioned therein. A lid is hingedly coupled to the box for opening and closing the box. A plurality of light emitters is each coupled to the lid and each of the light emitters emits light into the box. Each of the light emitters has an operational frequency in the ultra-violet spectrum of light to kill bacteria and sterilize the objects in the box. A lever extends through the outer wall of the box wherein the lever is configured to be manipulated by a user. The lever is positionable between an on position and an off position for turning the light emitters on and off.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a sterilization box assembly according to an embodiment of the disclosure.

FIG. 2 is a perspective view of an embodiment of the disclosure showing a lid in an open position.

FIG. 3 is a front view of an embodiment of the disclosure showing a lid in an open position.

FIG. 4 is a left side phantom view of an embodiment of the disclosure.

FIG. 5 is a schematic view of an embodiment of the disclosure.

### DETAILED DESCRIPTION OF THE INVENTION

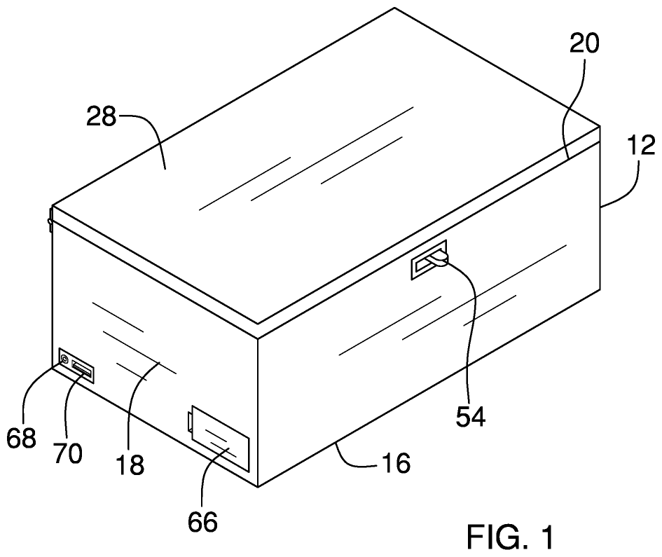

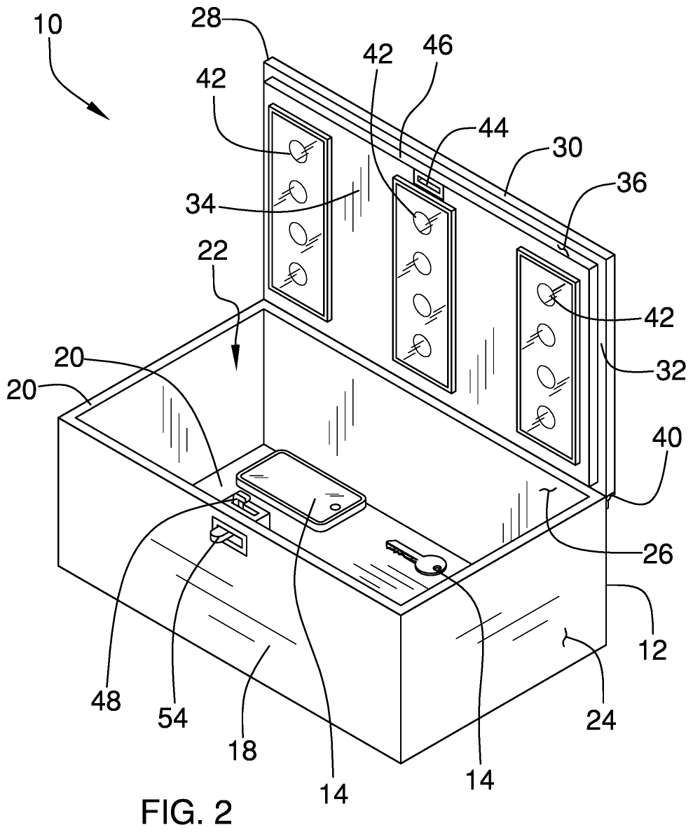

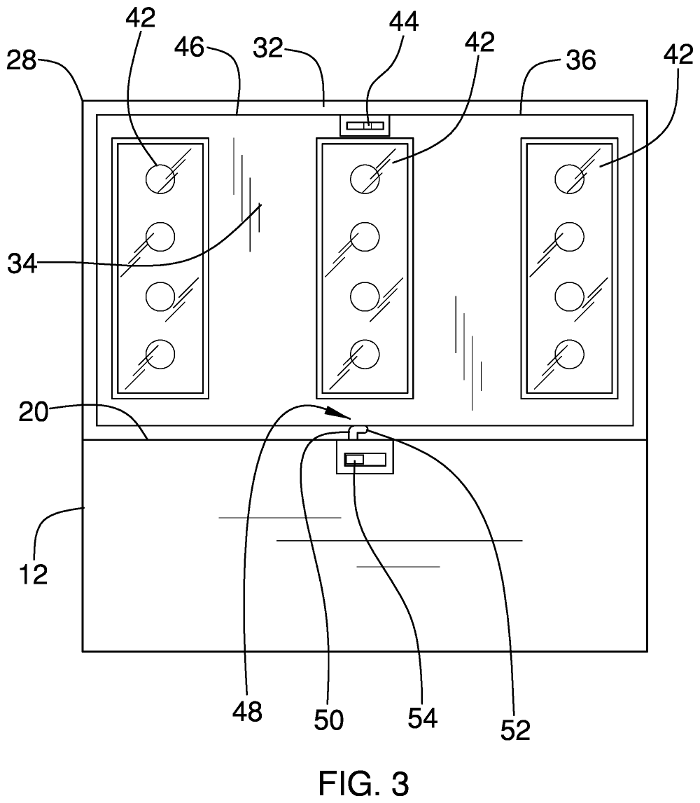

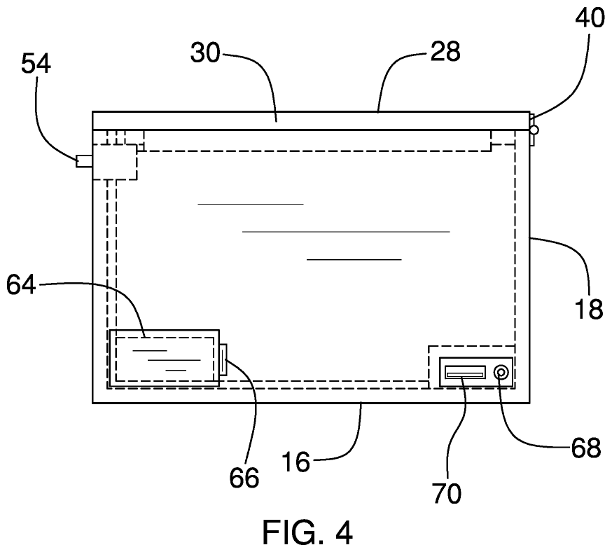

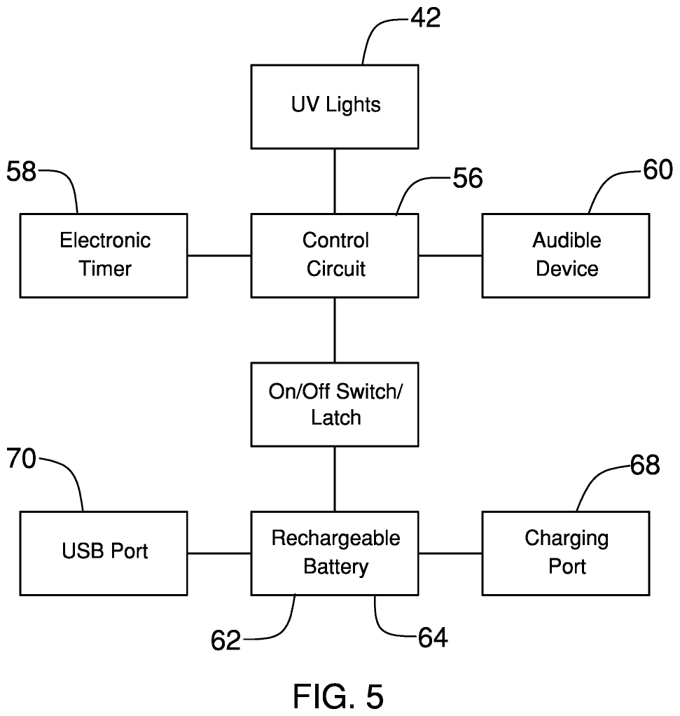

With reference now to the drawings, and in particular to FIGS. 1 through 5 thereof, a new sterilization device embodying the principles and concepts of an embodiment of the disclosure and generally designated by the reference numeral 10 will be described.

As best illustrated in FIGS. 1 through 5, the sterilization box assembly 10 generally comprises a box 12 that can have a variety of objects 14 positioned therein. The objects 14 might include a smart phone, jewelry, keys or any other object that has an unusual or irregular shape that would make it difficult to sanitize by hand. The box 12 is comprised of an opaque material to inhibit light from passing therethrough. The box 12 has a bottom wall 16 and an outer wall 18 extending upwardly therefrom, the outer wall 18 has a distal edge 20 with respect to the bottom wall 16 defining an opening 22 into an interior of the box 12, and the outer wall 18 has an outside surface 24 and an inside surface 26.

A lid 28 is provided and the lid 28 is hingedly coupled to the box 12 for opening and closing the box 12. The lid 28 is comprised of an opaque material to inhibit light from passing therethrough. The lid 28 has a perimeter edge 30 and bottom side 32, and the bottom side 32 has a prominence 34 extending away therefrom. The prominence 34 has a bounding surface 36 and a bottom surface 38, and the bounding surface 36 is spaced from the perimeter edge 30. The distal edge 20 of the outer wall 18 of the box 12 is positioned between the bounding surface 36 and the perimeter edge 30 when the lid 28 is closed. Additionally, the perimeter edge 30 has a back side 40, and the back side 40 is hingedly coupled to the outer wall 18 of the box 12.

A plurality of light emitters 42 is provided and each of the light emitters 42 is coupled to the lid 28. Each of the light emitters 42 is directed into the box 12 when the lid 28 is closed emit light into the box 12. Each of the light emitters 42 has an operational frequency in the ultra-violet spectrum of light. In this way each of the light emitters 42 can kill bacteria when the light emitters 42 are turned on thereby facilitating the objects 14 positioned within the box 12 to be sterilized. Each of the light emitters 42 is positioned on the bottom surface 38 of the prominence 34, and the light emitters 42 are spaced apart from each other and are distributed along a full width of the prominence 34. Each of the light emitters 42 may comprise an LED that emits ultraviolet light or other type of electronic light emitter.

An engagement 44 is recessed into the bottom surface 38 of the prominence 34 and the engagement 44 is positioned adjacent to a front side 46 of the bounding surface 36 of the prominence 34. The engagement 44 is centrally positioned along the front side 46. A latch 48 is provided and the latch 48 is integrated into the inside surface 26 of the outer wall 18 of the box 12. The latch 48 comprises a leg 50 extending upwardly on the outer wall 18 and a foot 52 extending laterally away from the leg 50. The foot 52 engages the engagement 44 when the lid 28 is closed and the latch 48 is in a locked position to inhibit the lid 28 from is opened. The foot 52 disengages the engagement 44 when the latch 48 is urged into an unlocked position.

A lever 54 extends through the outer wall 18 of the box 12 and the lever 54 can be manipulated by a user. The lever 54 engages the latch 48 and the lever 54 urges the latch 48 into the unlocked position when the lever 54 is positioned in an off position. Conversely, the lever 54 urges the latch 48 into the locked position when the lever 54 is positioned in an on position. A control circuit 56 is integrated into the box 12, the control circuit 56 is electrically coupled to each of the light emitters 42 and the lever 54 is electrically coupled to the control circuit 56. The light emitters 42 are turned on when the lever 54 is positioned in the on position. Conversely, the light emitters 42 are turned off when the lever 54 is positioned in the off position. The control circuit 56 receives an alert input.

An electronic timer 58 is integrated into the box 12 and the electronic timer 58 is electrically coupled to the control circuit 56. The electronic timer 58 is turned on when the lever 54 is positioned in the on position and the electronic timer 58 counts down a pre-determined duration of time when the timer is turned on. The control circuit 56 receives the alert input when the electronic timer 58 counts down the pre-determined duration of time, and the predetermined duration of time may be at least 5.0 minutes.

An audible alert 60 is integrated into the box 12 to emit an audible alarm outwardly therefrom. The audible alert 60 is electrically coupled to the control circuit 56 and the audible alert 60 is turned on when the control circuit 56 receives the alert input. In this way the audible alert 60 can alert a user to that the objects 14 in the box 12 have been sterilized. A power supply 62 is integrated into the box 12 and the power supply 62 is electrically coupled to the control circuit 56. The power supply 62 comprises a rechargeable battery 64 that is integrated into the box 12 and the rechargeable battery 64 is electrically coupled to the control circuit 56. The outer wall 18 of the box 12 has a battery cover 66 removably coupled thereto and the rechargeable battery 64 is positioned beneath the battery cover 66.

The power supply 62 includes a first charge port 68 that is recessed into the outer wall 18 of the box 12 and the first charge port 68 can be electrically coupled to a power source comprising a charger. The first charge port 68 is electrically coupled to the rechargeable battery 64 for charging the rechargeable battery 64. The power supply 62 includes a second charge port 70 that is recessed into the outer wall 18 of the box 12 to be electrically coupled to a power source comprising a charger. The second charge port 70 is electrically coupled to the rechargeable battery 64 for charging the rechargeable battery 64. The first charge port 68 may comprise a female 12.0 volt charge port and the second charge port 70 may comprise a usb port.

In use, the objects 14 are positioned in the box 12, the lid 28 closed and the lever 54 is positioned in the on position. Thus, the light emitters 42 are turned on to sterilize the objects 14 in the box 12. The audible alert 60 is turned on when the electronic timer 58 counts down the predetermined duration of time. In this way the user is alerted that the objects 14 have been sterilized and can be removed from the box 12. The lever 54 is moved to the off position to turn off the light emitters 42 and to facilitate the lid 28 to be opened.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sterilization box assembly for sterilizing a variety of objects that have shapes that are challenging to sterilize, said assembly comprising:
a box being configured to have a variety of objects positioned therein, said box being comprised of an opaque material wherein said box is configured to inhibit light from passing therethrough;
a lid being hingedly coupled to said box for opening and closing said box, said lid being comprised of an opaque material wherein said lid is configured to inhibit light from passing therethrough;
a plurality of light emitters, each of said light emitters being coupled to said lid, each of said light emitters being directed into said box when said lid is closed wherein each of said light emitters is configured to emit light into said box, each of said light emitters having an operational frequency in the ultra-violet spectrum of light wherein each of said light emitters is configured to kill bacteria when said light emitters are turned on thereby facilitating the objects positioned within said box to be sterilized;
a lever extends through said box wherein said lever is configured to be manipulated by a user, said lever being positionable between an on position and an off position, each of said light emitters being turned on when said lever is positioned in said on position, each of said light emitters being turned off when said lever is positioned in said off position.

2. The assembly according to claim 1, wherein said box has a bottom wall and an outer wall extending upwardly therefrom, said outer wall having a distal edge with respect to said bottom wall defining an opening into an interior of said box, said outer wall having an outside surface and an inside surface.

3. The assembly according to claim 2, wherein said lid has a perimeter edge and bottom side, said bottom side having a prominence extending away therefrom, said prominence having a bounding surface and a bottom surface, said bounding surface being spaced from said perimeter edge, said distal edge of said outer wall of said box being positioned between said bounding surface and said perimeter edge when said lid is closed, said perimeter edge having a back side, said back side being hingedly coupled to said outer wall of said box.

4. The assembly according to claim 3, wherein each of said light emitters is positioned on said bottom surface of said prominence, said light emitters being spaced apart from each other and being distributed along a full width of said prominence.

5. The assembly according to claim 3, further comprising an engagement being recessed into said bottom surface of said prominence, said engagement being positioned adjacent to a front side of said bounding surface of said prominence, said engagement being centrally positioned along said front side.

6. The assembly according to claim 5, further comprising a latch being integrated into said inside surface of said outer wall of said box, said latch comprising a leg extending upwardly on said outer wall and a foot extending laterally away from said leg, said foot engaging said engagement when said lid is closed and said latch is in a locked position to inhibit said lid from being opened, said foot disengaging said engagement when said latch is urged into an unlocked position.

7. The assembly according to claim 1, further comprising a control circuit being integrated into said box, said control circuit being electrically coupled to each of said light emitters, said lever being electrically coupled to said control circuit, said light emitters being turned on when said lever is positioned in said on position, said light emitters being turned off when said lever is positioned in said off position, said control circuit receiving an alert input.

8. The assembly according to claim 7, further comprising an electronic timer being integrated into said box, said electronic timer being electrically coupled to said control circuit, said electronic timer being turned on when said lever is positioned in said on position, said electronic timer counting down a pre-determined duration of time when said timer is turned on, said control circuit receiving said alert input when said electronic timer counts down said pre-determined duration of time.

9. The assembly according to claim 7, further comprising an audible alert being integrated into said box wherein said audible alert is configured to emit an audible alarm outwardly therefrom, said audible alert being electrically coupled to said control circuit, said audible alert being turned on when said control circuit receives said alert input wherein said audible alert is configured to alert a user to that the objects in said box have been sterilized.

10. The assembly according to claim 7, further comprising a power supply being integrated into said box, said power supply being electrically coupled to said control circuit, said power supply comprising:
a rechargeable battery being integrated into said box, said rechargeable battery being electrically coupled to said control circuit;
a first charge port being recessed into said outer wall of said box wherein said first charge port is configured to be electrically coupled to a power source comprising a charger, said first charge port being electrically coupled to said rechargeable battery for charging said rechargeable battery; and
a second charge port being recessed into said outer wall of said box wherein said second charge port is configured to be electrically coupled to a power source comprising a charger, said second charge port being electrically coupled to said rechargeable battery for charging said rechargeable battery.

11. A sterilization box assembly for sterilizing a variety of objects that have shapes that are challenging to sterilize, said assembly comprising:
a box being configured to have a variety of objects positioned therein, said box being comprised of an opaque material wherein said box is configured to inhibit light from passing therethrough, said box having a bottom wall and an outer wall extending upwardly therefrom, said outer wall having a distal edge with respect to said bottom wall defining an opening into an interior of said box, said outer wall having an outside surface and an inside surface;
a lid being hingedly coupled to said box for opening and closing said box, said lid being comprised of an opaque material wherein said lid is configured to inhibit light from passing therethrough, said lid having a perimeter edge and bottom side, said bottom side having a prominence extending away therefrom, said prominence having a bounding surface and a bottom surface, said bounding surface being spaced from said perimeter edge, said distal edge of said outer wall of said box being positioned between said bounding surface and said perimeter edge when said lid is closed, said perimeter edge having a back side, said back side being hingedly coupled to said outer wall of said box;
a plurality of light emitters, each of said light emitters being coupled to said lid, each of said light emitters being directed into said box when said lid is closed wherein each of said light emitters is configured to emit light into said box, each of said light emitters having an operational frequency in the ultra-violet spectrum of light wherein each of said light emitters is configured to kill bacteria when said light emitters are turned on thereby facilitating the objects positioned within said box to be sterilized, each of said light emitters being positioned on said bottom surface of said prominence, said light emitters being spaced apart from each other and being distributed along a full width of said prominence;
an engagement being recessed into said bottom surface of said prominence, said engagement being positioned adjacent to a front side of said bounding surface of said prominence, said engagement being centrally positioned along said front side;
a latch being integrated into said inside surface of said outer wall of said box, said latch comprising a leg extending upwardly on said outer wall and a foot extending laterally away from said leg, said foot engaging said engagement when said lid is closed and said latch is in a locked position to inhibit said lid from being opened, said foot disengaging said engagement when said latch is urged into an unlocked position;
a lever extending through said outer wall of said box wherein said lever is configured to be manipulated by a user, said lever engaging said latch, said lever urging said latch into said unlocked position when said lever is positioned in an off position, said lever urging said latch into said locked position when said lever is positioned in an on position;
a control circuit being integrated into said box, said control circuit being electrically coupled to each of said light emitters, said lever being electrically coupled to said control circuit, said light emitters being turned on when said lever is positioned in said on position, said light emitters being turned off when said lever is positioned in said off position, said control circuit receiving an alert input;
an electronic timer being integrated into said box, said electronic timer being electrically coupled to said control circuit, said electronic timer being turned on when said lever is positioned in said on position, said electronic timer counting down a pre-determined duration of time when said timer is turned on, said control circuit receiving said alert input when said electron timer counts down said pre-determined duration of time;
an audible alert being integrated into said box wherein said audible alert is configured to emit an audible alarm outwardly therefrom, said audible alert being electrically coupled to said control circuit, said audible alert being turned on when said control circuit receives said alert input wherein said audible alert is configured to alert a user to that the objects in said box have been sterilized; and
a power supply being integrated into said box, said power supply being electrically coupled to said control circuit, said power supply comprising:
a rechargeable battery being integrated into said box, said rechargeable battery being electrically coupled to said control circuit;
a first charge port being recessed into said outer wall of said box wherein said first charge port is configured to be electrically coupled to a power source comprising a charger, said first charge port being electrically coupled to said rechargeable battery for charging said rechargeable battery; and
a second charge port being recessed into said outer wall of said box wherein said second charge port is configured to be electrically coupled to a power source comprising a charger, said second charge port being electrically coupled to said rechargeable battery for charging said rechargeable battery.
